(19) United States
(12) Patent Application Publication
(10) Pub. No.: US 2026/0294654 A1
(43) Pub. Date: Oct. 1, 2026

(54) CRIMPING METHOD AND CRIMPING DEVICE FOR A TUBULAR STENT, AND IMPLANTABLE DEVICE

(71) Applicant: TERUMO KABUSHIKI KAISHA, Tokyo (JP)

(72) Inventor: Risa SEKIGUCHI, Hadano Kanagawa (JP)

(21) Appl. No.: 19/676,814

(22) Filed: May 14, 2026

Related U.S. Application Data
(63) Continuation of application No. PCT/JP2024/044371, filed on Dec. 16, 2024.

(30) Foreign Application Priority Data
Dec. 26, 2023 (JP) 2023-218896

(51) Int. Cl. A61F 2/89; A61F 2/95; A61F 2/958; A61M 25/10

(52) U.S. Cl. CPC A61F 2/89; A61F 2/9524; A61F 2/958; A61M 25/1027; A61F 2210/0076; A61F 2220/0025; A61F 2230/0069; A61F 2250/0024; A61F 2250/0067; A61M 2207/00

(57) ABSTRACT

A method for crimping a tubular stent onto a balloon using one or more pressing members includes arranging the stent on an outer circumferential side of the balloon, heating the one or more pressing members, reducing a diameter of the stent by pressing the one or more heated pressing members against a porous structure that covers an outer circumference of the stent, from an outer surface side of the porous structure, and pressurizing an inside of the balloon at least once.

### CROSS-REFERENCE TO RELATED APPLICATION

This application is a continuation of International Patent Application No. PCT/JP2024/044371 filed Dec. 16, 2024, which is based upon and claims the benefit of priority from Japanese Patent Application No. 2023-218896 filed Dec. 26, 2023, the entire contents of which are incorporated herein by reference.

### TECHNICAL FIELD

The present disclosure relates to a crimping method and a crimping device for a tubular stent, and an implantable device.

### BACKGROUND ART

A stent is known as a medical device used for treating a lesion site where stenosis or occlusion has occurred in a biological lumen such as a blood vessel. Various types of stents exist. Among them, there is a so-called “balloon-expandable stent (balloon-expandable stent delivery system)” that is delivered to a lesion site in a state of being crimped onto a balloon of a balloon catheter, and that is implanted at the lesion site as the balloon is expanded after being delivered to the lesion site.

In a manufacturing method for the above-described balloon-expandable stent delivery system (hereinafter also simply referred to as a “stent delivery system”), a crimping step of crimping (holding) a stent on an outer circumferential surface of a balloon is performed.

The crimping step is performed, for example, using a conventional crimping device. Such a crimping device includes a plurality of pressing members (crimp heads) configured to be movable toward and away from a balloon and a stent arranged on the outer circumferential surface of the balloon. In the crimping step performed using the crimping device, the balloon and the stent are arranged near a central position in an approaching direction of the pressing members, and each of the plurality of pressing members is moved toward the stent from an outer circumferential side toward a radially inward direction while applying a pressing force so as to press the stent against the balloon, thereby crimping the stent onto the outer circumferential surface of the balloon. In addition, in the crimping step using the crimping device, while the plurality of pressing members are being pressed against the stent from the outer circumferential side as described above, an inside of the balloon is pressurized to cause the balloon to be pinched into gaps between struts of the stent, thereby further improving a crimping force of the stent with respect to the balloon and reducing a profile after crimping.

### SUMMARY

In order to reduce a profile of a stent, it is conceivable to increase a pressing force of pressing members during crimping (a crimp load of crimp heads). For example, in the related art, a method of crimping while pressurizing a balloon as in the conventional crimping step has often been employed. However, in this method, when the pressing force of the pressing members increases, a force with which the stent pinches the balloon becomes excessively strong, thereby increasing a possibility that a pinhole is formed in the balloon. On the other hand, in a method of crimping without pressurizing the balloon, it is possible to reduce an occurrence rate of pinholes, but it is difficult to improve a crimping force, and it is also difficult to reduce the profile.

Embodiments of the present disclosure provide a crimping method and a crimping device for a tubular stent, and an implantable device that are capable of improving a crimping force and reducing a profile while reducing the occurrence rate of pinholes.

In one embodiment, a method for crimping a tubular stent onto a balloon using one or more pressing members, comprises: arranging the stent on an outer circumferential side of the balloon; heating the one or more pressing members; reducing a diameter of the stent by pressing the one or more heated pressing members against a porous structure that covers an outer circumference of the stent, from an outer surface side of the porous structure; and pressurizing an inside of the balloon at least once.

According to the above-described method, it is possible to improve a crimping force of the implantable device and reduce a profile of the implantable device while reducing the occurrence rate of pinholes.

### BRIEF DESCRIPTION OF DRAWINGS

FIG. 1 is a schematic plan view illustrating a stent delivery system including an implantable device according to an embodiment.

FIG. 2 is a schematic plan view illustrating a reduced-diameter state of a stent and a porous structure of the implantable device.

FIG. 3 is a schematic plan view illustrating an expanded-diameter state of the stent and the porous structure.

FIG. 4 is a partially enlarged view illustrating a part of the stent and the porous structure in the expanded-diameter state.

FIG. 5 is a partially enlarged view illustrating a part of the stent and the porous structure in the reduced-diameter state.

FIG. 6 is an enlarged view of a portion indicated by a broken line in FIG. 5.

FIG. 7 is a cross-sectional view of the stent and the porous structure taken along arrow 7A-7A in FIG. 6.

FIG. 8 is a view schematically illustrating a part of a drug coating layer provided on the stent.

FIG. 9 is an axial orthogonal cross-sectional view taken along arrow 9A-9A in FIG. 1.

FIG. 10 is a perspective view illustrating a crimping device according to an embodiment.

FIG. 11 is a view illustrating a flowchart of a crimping method according to an embodiment.

FIG. 12 is a view for describing the crimping method and illustrating a state in which a preparation item before forming the implantable device is set in the crimping device.

FIG. 13 is a view for describing the crimping method.

FIG. 14 is a view for describing the crimping method.

FIG. 15 is a view for describing the crimping method.

### DESCRIPTION OF EMBODIMENTS

Hereinafter, embodiments of the present disclosure will be described with reference to the accompanying drawings. Note that the following description does not limit a technical scope or meanings of terms described in the claims. Further, dimensional ratios in the drawings are exaggerated for convenience of explanation and may differ from actual ratios.

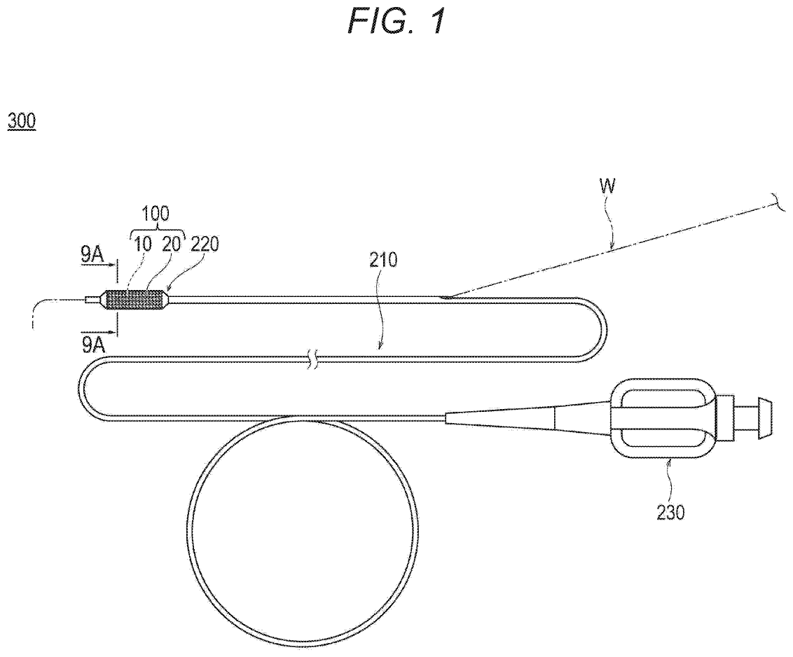

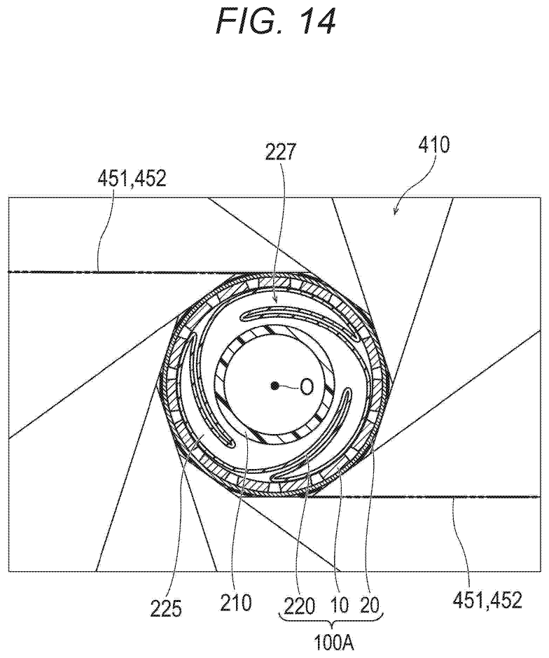

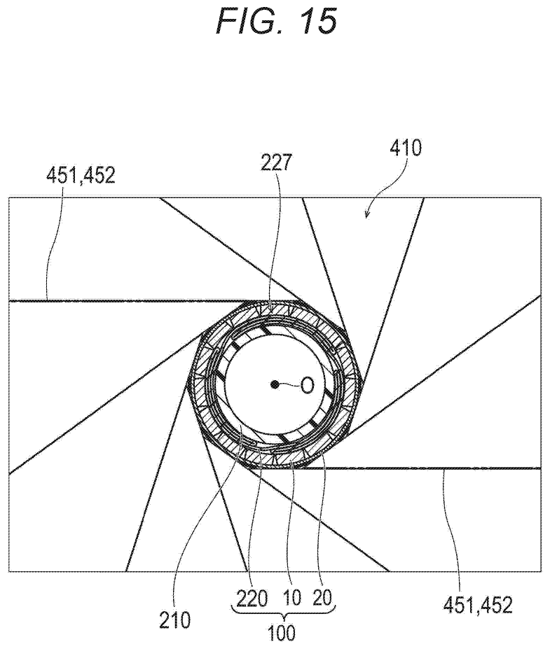

Embodiments will be described with reference to FIGS. 1 to 15.

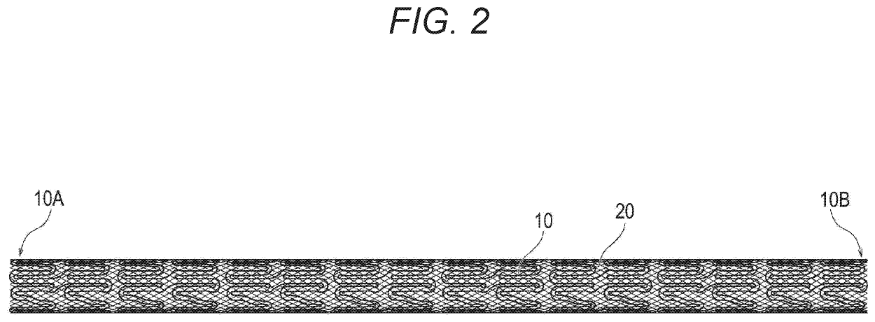

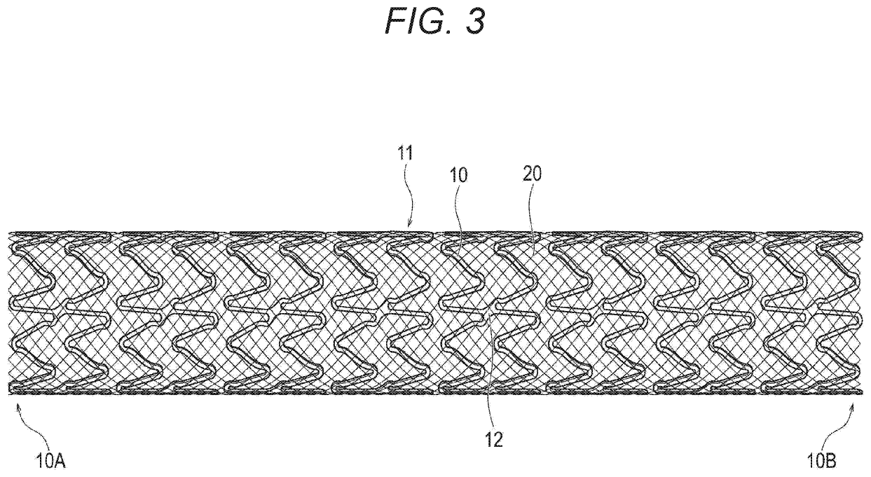

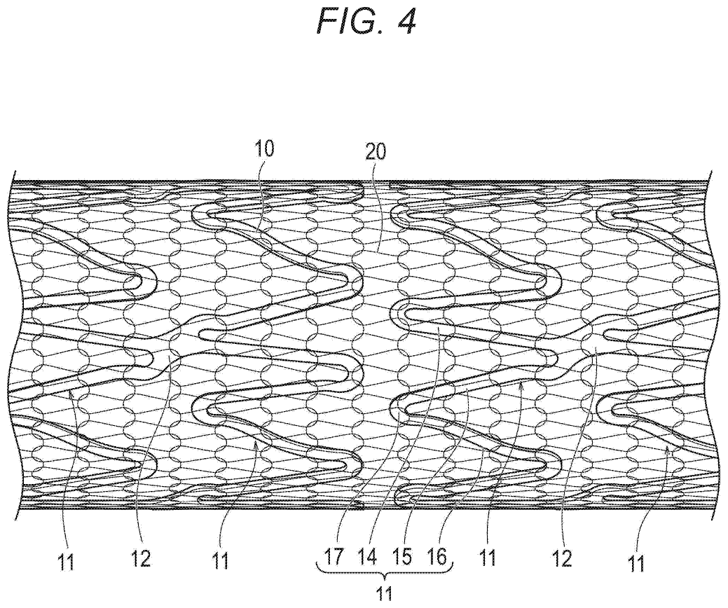

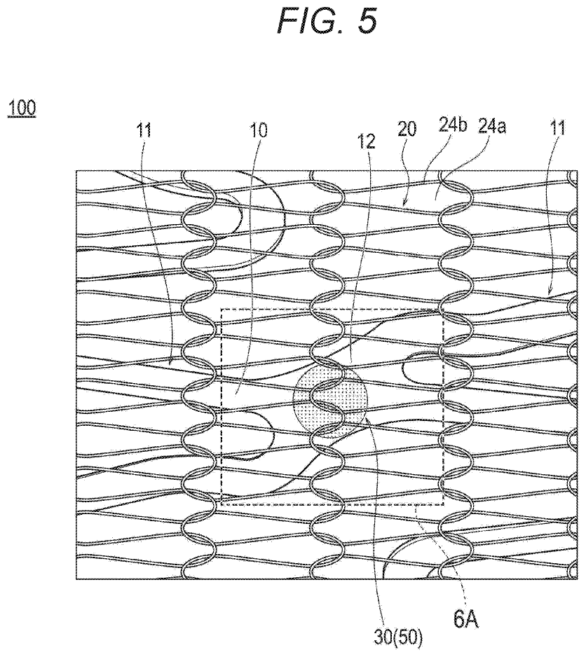

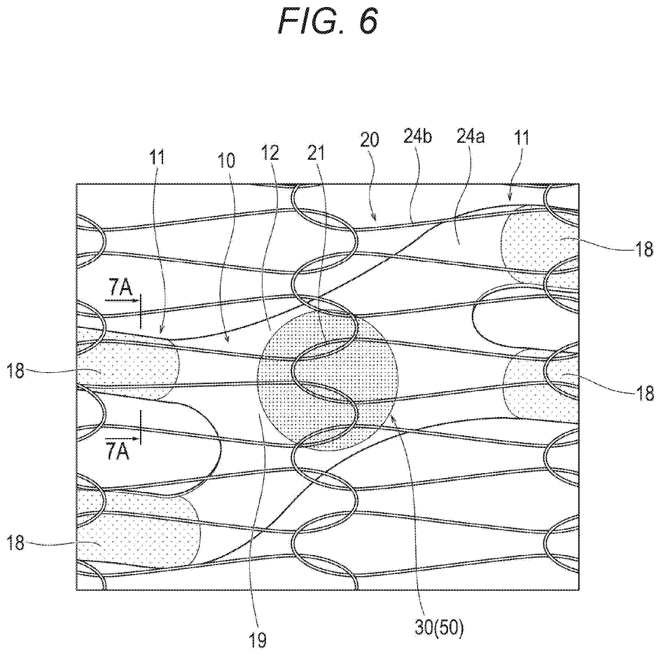

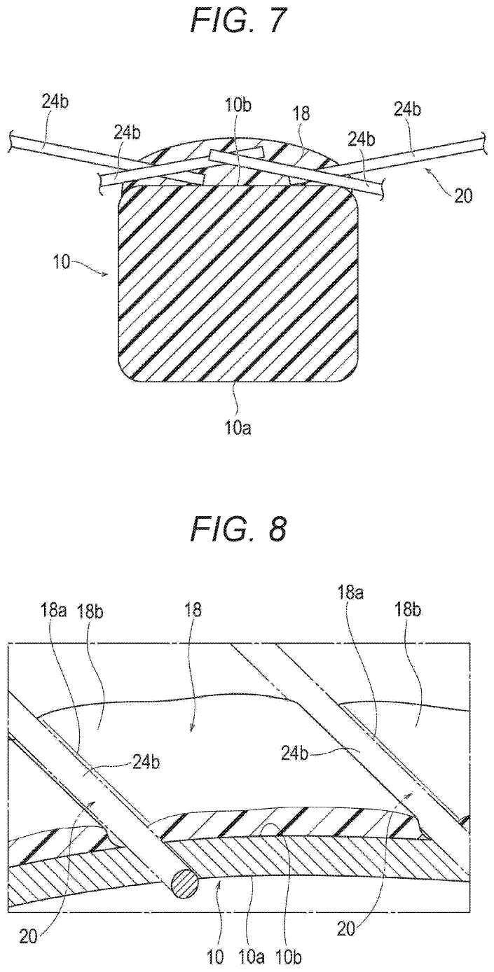

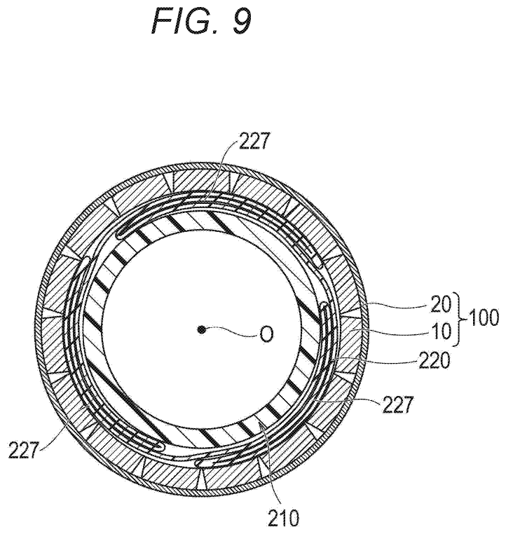

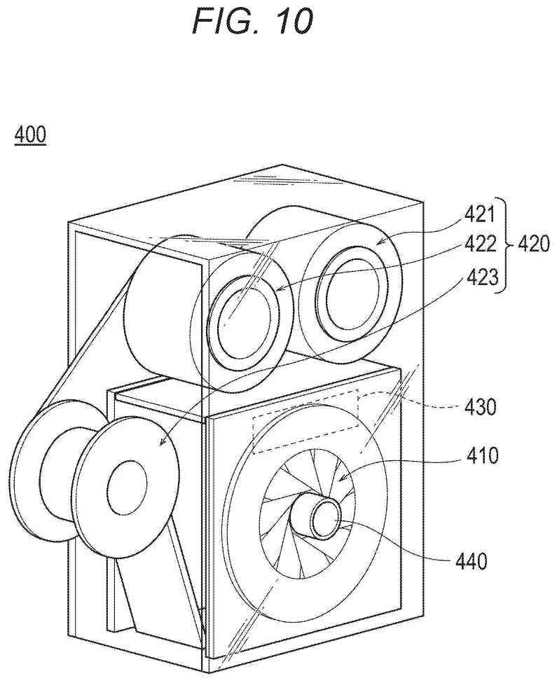

FIG. 1 is a view illustrating a stent delivery system 300 including an implantable device 100 according to an embodiment. FIGS. 2 to 9 are views for describing the implantable device 100, a stent 10, and a porous structure 20. FIG. 2 is a schematic plan view illustrating a reduced-diameter state of the stent 10 and the porous structure 20. FIG. 3 is a schematic plan view illustrating an expanded-diameter state of the stent 10 and the porous structure 20, and FIG. 4 is a view illustrating an enlarged part of the stent 10 and the porous structure 20 illustrated in FIG. 3. FIG. 5 is a view illustrating a further enlarged part of the stent 10 and the porous structure 20, and FIG. 6 is an enlarged view of a portion 6A indicated by a broken line in FIG. 5. FIG. 7 is a cross-sectional view of the stent 10 and the porous structure 20 taken along arrow 7A-7A in FIG. 6. FIG. 8 is a view schematically illustrating a part of a drug coating layer 18 provided on the stent 10. FIG. 9 is an axial orthogonal cross-sectional view taken along arrow 9A-9A in FIG. 1 (an axial orthogonal cross-sectional view of the implantable device 100 in a reduced-diameter state).

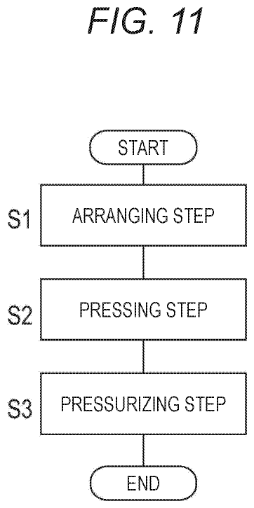

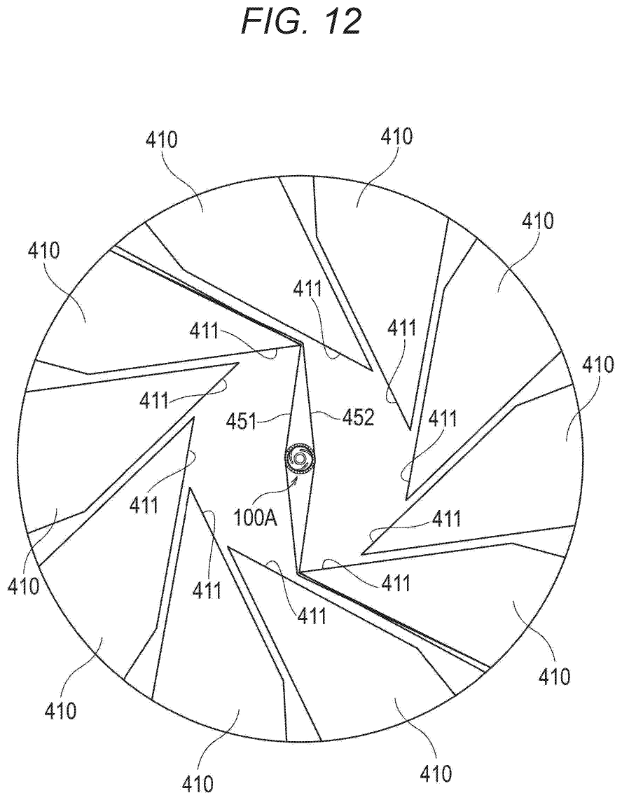

In addition, FIG. 10 is a view illustrating a crimping device 400. FIG. 11 is a flowchart illustrating respective steps of a crimping method, and FIGS. 12 to 15 are views for describing the crimping method.

In the present specification, a longitudinal direction in which the stent 10 extends is referred to as an “axial direction”, a direction orthogonal to the axial direction (a direction in which the implantable device 100 and a catheter body portion 210 approach and separate from a central axis O in the axial orthogonal cross-sectional view in FIG. 9) is referred to as a “radial direction”, and a rotational direction with reference to the central axis O (a clockwise direction and a counterclockwise direction in the axial orthogonal cross-sectional view in FIG. 9) is referred to as a “circumferential direction (outer circumferential direction or inner circumferential direction)”. In addition, in the stent 10, a side inserted into a living body is referred to as a “distal side”, and a side that is opposite to the distal side and on which an operator operates the stent delivery system 300 is referred to as a “proximal side”.

### <Implantable Device 100>

The implantable device 100 is arranged on an outer circumference of a balloon 220 that is expandable and contractible, as illustrated in FIG. 1. As illustrated in FIGS. 2 and 9, the implantable device 100 includes a tubular stent 10 that is expandable in diameter and is crimped onto an outer circumferential surface of the balloon 220, and a porous structure 20 that is arranged to cover the stent 10 and of which at least a part is fixed to the stent 10.

As illustrated in FIGS. 6 and 7, the stent 10 has a drug coating layer 18 arranged on a surface of the stent 10, the surface facing the porous structure 20 (an outer surface 10b that faces an outside of the stent 10). An inner surface 10a of the stent 10 that faces an inside is not provided with the drug coating layer 18.

In FIG. 6, a part of the drug coating layer 18 formed on the outer surface 10b of the stent 10 is illustrated in a plan view. In the present specification, illustration of the drug coating layer 18 is omitted in drawings other than FIGS. 6, 7, and 8 for convenience of illustration.

The stent 10 and the porous structure 20 constitute the implantable device 100 together with the balloon 220 in a state of being arranged on the outer circumferential surface of the balloon 220 of a balloon catheter 200 (in a state in which the stent 10 is crimped onto the balloon 220).

The balloon catheter 200 to which the implantable device 100 is mounted constitutes the stent delivery system 300. The stent delivery system 300 delivers the implantable device 100 to a lesion site in a contracted state, and expands the stent 10 and the porous structure 20 in accordance with expansion of the balloon 220, thereby allowing the stent 10 and the porous structure 20 to be implanted at the lesion site.

The balloon catheter 200 includes an elongated catheter body portion 210, a balloon 220 provided at a distal end of the catheter body portion 210, and a hub 230 fixed to a proximal end of the catheter body portion 210.

The balloon catheter 200 is configured as a rapid exchange type balloon catheter that allows a guidewire W to be introduced from a vicinity of a distal end portion of the catheter body portion 210 and allows the guidewire W to be inserted toward a distal side of the balloon 220. The balloon catheter 200 may also be configured as a so-called over-the-wire type balloon catheter.

As a material forming the balloon 220, for example, an organic polymer material can be used. Specifically, as the material, it is possible to use polymer materials such as polyolefins (for example, polyethylene, polypropylene, polybutene, an ethylene-propylene copolymer, an ethylene-vinyl acetate copolymer, an ionomer, or a mixture of two or more thereof), polyvinyl chloride, polyamide, a polyamide elastomer, polyurethane, a polyurethane elastomer, polyimide, and a fluororesin, or mixtures thereof, or elastic resin materials including two or more of the above polymer materials, and among these, a polyamide-based resin can be suitably used as a main material.

The implantable device 100 will be described in detail.

The implantable device 100 is used for treating a stenosis or an occlusion that has formed in a blood vessel, a bile duct, a trachea, an esophagus, a urethra, or another biological lumen. The stent 10 used in the implantable device 100 is formed as a so-called balloon-expandable medical device that is arranged in a state of being crimped onto the folded balloon 220, is expanded after being delivered to a lesion site, and is implanted at the lesion site.

As illustrated in FIGS. 2 to 4, the stent 10 has a cylindrical shape extending in the axial direction. The porous structure 20 is arranged to cover the outer circumference of the stent 10 and has a cylindrical shape similar to that of the stent 10.

As illustrated in FIGS. 2, 3, 4, and 5, the stent 10 includes a distal end 10A, a proximal end 10B, and a fixing portion 30 that fixes the porous structure 20 to the stent 10.

The stent 10 is radially expandable (to the state illustrated in FIGS. 3 and 4) and contractible (to the state illustrated in FIG. 2).

As illustrated in FIGS. 3 to 6, the stent 10 includes linear rings 11 that form an outer circumference of a cylindrical shape in which gaps are formed, and link portions 12 that connect the rings 11 to each other in gaps defined between the linear rings 11 adjacent in the axial direction.

The rings 11 extend in the circumferential direction of the stent 10 in a wave-shaped pattern that reciprocates with respect to the axial direction. As illustrated in FIG. 4, each of the wave-shaped rings 11 includes a plurality of first strut portions 15 formed of a straight line or a curved line, a plurality of second strut portions 16 formed of a straight line or a curved line, and a plurality of curved portions 17 formed between the first strut portions 15 and the second strut portions 16. In addition, as illustrated in FIG. 4, the rings 11 include a plurality of third strut portions 14 formed of a straight line or a curved line, the third strut portions 14 being arranged in pairs in the circumferential direction and being adjacent to the link portion 12 on one side in the axial direction.

The rings 11 are arranged in sequence along the axial direction, and adjacent rings 11 in the axial direction are integrated by the link portions 12. Accordingly, the stent 10 having a desired length can be easily obtained by increasing or decreasing the number of the rings 11.

As illustrated in FIGS. 6 and 7, the drug coating layer 18 can be provided on at least a part of the outer surface 10b of the stent 10. For example, the drug coating layer 18 can be arranged on the first strut portions 15 and the third strut portions 14 of the rings 11 illustrated in FIG. 4.

In the example shown in FIGS. 6 and 7, the drug coating layer 18 is not provided on the link portions 12. A portion in a predetermined range including the link portions 12 where the drug coating layer 18 is not provided constitutes an exposed portion 19. Specifically, the curved portions 17 of the rings 11 and the link portions 12 (portions where stress is concentrated and/or strain is produced due to expansion deformation) constitute the exposed portions 19. Since the drug coating layer 18 is not formed on the curved portions 17 of the rings 11 and the link portions 12, it is possible to avoid peeling or detachment of the drug coating layer 18 caused by stress being concentrated on the drug coating layer 18 and bending or strain being produced when the stent 10 expands in diameter.

A drug coated on the outer surface 10b of the stent 10 is supported by a polymer to constitute the drug coating layer 18. The polymer is preferably a biodegradable polymer. In this case, after the stent 10 is implanted in a living body, the drug is released in a sustained manner while the polymer is biodegraded, thereby more reliably preventing restenosis at a stent implantation site in the living body and suppressing inflammation derived from the polymer.

The biodegradable polymer described above is, for example, at least one polymer selected from the group consisting of polyester, aliphatic polyester, polyanhydride, polyorthoester, polycarbonate, polyphosphazene, polyphosphate ester, polyvinyl alcohol, polypeptide, polysaccharide, protein, and cellulose, a copolymer obtained by optionally copolymerizing monomers constituting the polymer, and a mixture of the polymer and/or the copolymer. The aliphatic polyester is, for example, polylactic acid (PLA), polyglycolic acid (PGA), or a lactic acid-glycolic acid copolymer (PLGA).

Note that a primer coating layer (not illustrated) may be arranged between the drug coating layer 18 and the outer surface 10b of the stent 10. A primer constituting the primer coating layer can be appropriately selected in consideration of adhesiveness to the polymer contained in the drug coating layer 18 and adhesiveness to the outer surface 10b of the stent 10. By providing the primer coating layer, peeling resistance of the drug coating layer 18 can be improved.

The stent 10 can be formed of a metal material or a polymer material. When the stent 10 is formed of a metal material, the material is not particularly limited, and a metal material generally used for a known stent can be used. Specifically, examples include stainless steels such as SUS304, SUS316, SUS316L, SUS420J2, and SUS630, tantalum, titanium, nickel-titanium alloys, tantalum-titanium alloys, nickel-aluminum alloys, Inconel, gold, platinum, iridium, tungsten, and cobalt-based alloys such as cobalt-chromium (Co—Cr) alloys.

When the stent 10 is formed of a polymer material, the material is not particularly limited, and a polymer material generally used for a known stent can be used. Specifically, examples include polyolefins such as polyethylene and polypropylene, aromatic polyesters such as polyethylene terephthalate, cellulose-based polymers such as cellulose acetate and cellulose nitrate, and fluoropolymers such as polytetrafluoroethylene and a tetrafluoroethylene-ethylene copolymer.

The porous structure 20 is arranged to cover the outer circumference of the stent 10 and is expandable in diameter following expansion of the stent 10.

As illustrated in FIGS. 5 and 6, the porous structure 20 includes void portions 24a penetrating through the porous structure 20 in a thickness direction and framework portions 24b that define the void portions 24a.

The implantable device 100 includes the porous structure 20 arranged on the outer circumference of the stent 10, thereby allowing the porous structure 20 to be biased against a blood vessel in accordance with expansion of the stent 10 when the stent 10 is expanded within the blood vessel. Accordingly, the implantable device 100 can prevent the porous structure 20 from being displaced from an implantation site or being shifted in position due to pressure of blood flow within the blood vessel.

The porous structure 20 has a cylindrical shape extending in the axial direction and has a mesh structure including the void portions 24a and the framework portions 24b.

As illustrated in FIGS. 4, 5, and 6, gaps in the mesh structure of the porous structure 20 are smaller than gaps between adjacent rings 11 of the stent 10. According to this configuration, when the stent 10 is expanded in diameter to implant the stent 10 at a lesion site, peripheral embolism can be prevented.

The size of each of the plurality of void portions 24a included in the porous structure 20 preferably is smaller in area than the gap between the rings 11 of the stent 10. By adopting such a configuration, scattering of plaque or thrombi due to expansion of the stent 10 can be prevented. On the other hand, the size of each of the void portions 24a is preferably larger than the area of a single blood cell contained in blood. This allows blood cells to pass through the void portions 24a. Furthermore, the void portions 24a impart extensibility to the porous structure 20, and thus, when the stent 10 expands in diameter, the porous structure 20 also expands in the circumferential direction in accordance with the expansion of the stent 10, thereby exhibiting good followability (expandability) with respect to expansion of the stent 10.

The porous structure 20 can be formed, for example, of a knitted fabric (knit), a woven fabric (braid), or a molded product (a member such as a film in which slits are formed). In a case where the porous structure 20 is a knitted fabric, the porous structure 20 can be formed by weft knitting. By forming the porous structure 20 by weft knitting, it is possible to suppress shortening of the length of the porous structure 20 in the axial direction accompanying expansion of the stent 10. In a case where the porous structure 20 is a woven fabric, the woven fabric is formed by a known weaving method. The porous structure 20 may also be formed of a molded product in which holes are formed in a tubular body formed by injection molding or the like. The size, shape, and number of meshes and weaves of the porous structure 20 are not particularly limited as long as peripheral embolism during expansion of the stent 10 can be prevented.

The material forming the porous structure 20 is not particularly limited, and may be, for example, a metal or a polymer.

As a metal forming the porous structure 20, for example, the metals exemplified above as materials forming the stent 10 can be used.

As a polymer forming the porous structure 20, for example, the following materials can be used. (1) A polymer selected from the group consisting of aliphatic polyester, polyester, polyanhydride, polyorthoester, polycarbonate, polyphosphazene, polyphosphate ester, polyvinyl alcohol, polypeptide, polysaccharide, protein, and cellulose; and (2) a copolymer formed of two or more monomers forming the polymer described in (1) are exemplified. Here, the aliphatic polyester is not particularly limited, and examples thereof include polylactic acid (PLA) such as poly-L-lactic acid (PLLA), poly-D-lactic acid (PDLA), and poly-DL-lactic acid (PDLLA), polyglycolic acid (PGA), polyhydroxybutyric acid, polyhydroxyvaleric acid, polyhydroxypentanoic acid, polyhydroxyhexanoic acid, polyhydroxyheptanoic acid, poly(ϵ-caprolactone) (PCL), polytrimethylene carbonate, poly 2,2-dimethyltrimethylene carbonate, polydioxanone, polybutyrolactone, polyvalerolactone, polymalic acid, polyethylene adipate, polyethylene succinate, polybutylene adipate, and polybutylene succinate. The polycarbonate is not particularly limited, and examples thereof include tyrosine-derived polycarbonate (Tyrosine-polycarbonate). In addition, polymers other than the above-described biodegradable polymers can also be used.

A fiber diameter of the porous structure 20 (which is synonymous with an outer diameter of the framework portions 24b) is not particularly limited, and may be, for example, 20 μm.

FIG. 8 schematically illustrates a state of the drug coating layer 18 in a state where the stent 10 is crimped onto the balloon 220. In FIG. 8, the framework portions 24b of the porous structure 20 are illustrated by two-dot chain lines in order to simplify the illustration.

As illustrated in FIG. 8, the drug coating layer 18 provided on the stent 10 has recesses 18a and protrusions 18b corresponding to the void portions 24a and the framework portions 24b of the porous structure 20. The drug coating layer 18 is depressed such that portions corresponding to the framework portions 24b (portions where the framework portions 24b are pressed) form the recesses 18a when the porous structure 20 is pressed against an outer surface side of the drug coating layer 18 by a crimping method described later. When the framework portions 24b are pressed, a part of the drug coating layer 18 originally present in the recesses 18a moves to surrounding regions. As a result, the protrusions 18b are formed at positions adjacent to the recesses 18a. As described in the crimping method described later, in the implantable device 100, when the stent 10 is crimped onto the balloon 220, the porous structure 20 is pressed against the drug coating layer 18 so as to form the recesses 18a, thereby allowing a uniform pressing force to be applied in the circumferential direction of the stent 10. As a result, it is possible to suitably prevent formation of pinholes in the balloon 220.

At least a part of the porous structure 20 is arranged in a state of being embedded in the recesses 18a of the drug coating layer 18. That is, the porous structure 20 is pressed against the drug coating layer 18 to form the recesses 18a and the protrusions 18b in the drug coating layer 18, and the framework portions 24b are arranged in a state of being embedded in (entering into) the recesses 18a. In the implantable device 100, the framework portions 24b of the porous structure 20 are embedded in the drug coating layer 18 in a state in which the stent 10 is crimped onto the balloon 220, and therefore, the profile of the implantable device 100 is reduced.

The fixing portion 30 of the stent 10 can be provided to fix at least a part of the porous structure 20 to the stent 10 at a position excluding the distal end 10A and the proximal end 10B of the stent 10, for example. In the present specification, the distal end 10A of the stent 10 refers to a “region at the most distal end of the stent 10 where the link portion 12 arranged at the most distal end is not provided”. In addition, in the present specification, the proximal end 10B of the stent 10 refers to a “region at the most proximal end of the stent 10 where the link portion 12 arranged at the most proximal end is not provided”.

The fixing portion 30 can be formed of a weldable fixing material 50. For example, by arranging the fixing material 50 at a portion of the stent 10 where the fixing portion 30 is provided and embedding a part of the porous structure 20 in the fixing material 50, the porous structure 20 can be fixed to the stent 10.

As the fixing material 50 used for the fixing portion 30, for example, a polymer exemplified above as a material forming the porous structure 20 can be used. As the fixing material 50, a biodegradable polymer is particularly preferably used. According to this configuration, since the fixing material 50 is biodegraded in a living body after implantation, inflammation derived from the polymer can be suppressed. The fixing material 50 may also be, for example, wax.

The fixing portion 30 can be provided, for example, at all of the link portions 12 included in the stent 10. The link portions 12 have a small change in axial geometry accompanying expansion of the stent 10. Therefore, at the link portions 12, when the stent 10 expands in diameter, tension of the porous structure 20 accompanying expansion of the stent 10 does not occur, and peeling or breakage of the porous structure 20 due to the fixing portions 30 provided at the link portions 12 can be suitably suppressed.

As described above, the drug coating layer 18 is not provided on the link portions 12. Therefore, as illustrated in FIG. 6, in the fixing portion 30, the porous structure 20 is directly fixed to the stent 10 at the exposed portions 19 of the stent 10 where the drug is not applied. In other words, the fixing material 50 forming the fixing portion 30 is directly fixed to the link portions 12 of the stent 10. According to this configuration, compared with a configuration in which the fixing material 50 is fixed to the stent 10 via the drug coating layer 18, it is possible to improve a fixing strength of the porous structure 20 to the stent 10 when the fixing material 50 is fixed (welded).

The fixing portion 30 is configured such that the porous structure 20 is directly fixed to the stent 10 at the exposed portions 19 of the stent 10 where the drug is not applied. Accordingly, even when the fixing portion 30 is welded by heating, it is possible to prevent deterioration of efficacy of the drug because the drug coating layer 18 is not present.

When the fixing material 50 is fixed to the stent 10 with the drug coating layer 18 interposed therebetween, it is preferable to employ a method without heating, such as an adhesive, or a method with a small influence of heating.

As illustrated in FIG. 6, loop portions 21 of a mesh knitted along the circumferential direction of the porous structure 20 can be fixed to the stent 10. It is preferable that at least the loop portions 21 are included in portions fixed by the fixing portion 30. According to this configuration, since the loop portions 21 extending along the circumferential direction can be fixed to the stent 10, a long length of the porous structure 20 can be embedded in the fixing material 50, and fixing strength can be improved.

### <Crimping Device 400>

FIG. 10 is a schematic perspective view illustrating the crimping device 400. FIGS. 12 to 15 are simplified front views illustrating a part of the crimping device 400 (a part including a pressing member 410) when a crimping method is performed.

The crimping device 400 is used for crimping the tubular stent 10 that is expandable in diameter onto the balloon 220.

The crimping device 400 includes one or more pressing members (crimp heads) 410 that crimp the stent 10 onto the balloon 220 arranged on the inner circumferential side of the stent 10 by reducing a diameter of the stent 10 in accordance with movement in a radially inward direction, a supply unit 420 that supplies sheet-like members 451 and 452 between the pressing members 410 and the stent 10, and a heating unit 430 that heats the pressing members 410.

As illustrated in FIGS. 10 and 12, the crimping device 400 includes a setting portion 440 in which respective members before crimping (the balloon 220, the stent 10, and the porous structure 20; hereinafter referred to as “preparation item 100A”) are set.

As illustrated in FIGS. 12 to 15, the pressing members 410 are arranged so as to surround the preparation item 100A set in the setting portion 440. In the example shown in FIGS. 12 to 15, the crimping device 400 includes ten pressing members 410. However, the number of the pressing members 410 included in the crimping device 400 is not particularly limited.

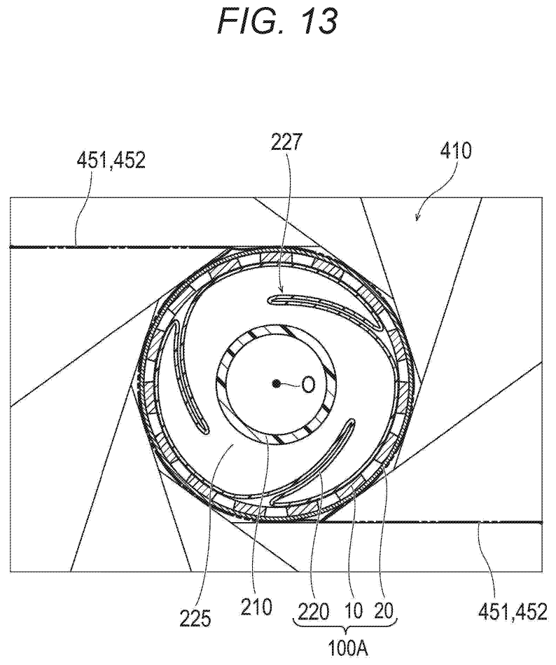

As illustrated in FIGS. 12 to 15, each of the pressing members 410 approaches the preparation item 100A set in the setting portion 440 when the crimping method is performed. Each of the pressing members 410 gradually reduces a space defined on an inner side (radially inward side) in a moving direction of the pressing members 410 as the pressing members 410 approach the preparation item 100A. Each of the pressing members 410 is provided with a pressing end 411 that applies a pressing force to the preparation item 100A during crimping. When the pressing members 410 approach the preparation item 100A, the pressing ends 411 are pressed against the preparation item 100A while a space defined inside the pressing ends 411 forms a substantially circular shape. In this state, by further moving each of the pressing members 410 in the radially inward direction, it is possible to increase the pressing force applied to the preparation item 100A in accordance with reduction in diameter of a space defined inside the pressing ends 411. In FIGS. 13 to 15, the sheet-like members 451 and 452 used in the crimping device 400 are illustrated in a simplified manner by two-dot chain lines.

The heating unit 430 heats each of the pressing members 410 to a predetermined temperature when the preparation item 100A is crimped. The heating unit 430 can be formed, for example, of a known heater or the like.

The supply unit 420 can be formed of a plurality of supply rollers 421, 422, and 423 that supply the sheet-like members 451 and 452 between the outer circumference of the preparation item 100A set in the setting portion 440 and each of the pressing members 410. For example, as illustrated in FIG. 12, when the preparation item 100A is crimped, the two sheet-like members 451 and 452 are supplied between the preparation item 100A and the pressing ends 411 of the respective pressing members 410 so as to pinch the preparation item 100A. The crimping device 400 applies a pressing force from the outer surface side of the porous structure 20 positioned at the outermost side of the preparation item 100A by moving each of the pressing members 410 toward the preparation item 100A in a state in which the sheet-like members 451 and 452 are arranged between the pressing members 410 and the preparation item 100A.

The sheet-like members 451 and 452 can be formed, for example, of a known resin sheet (polymer film) such as PTFE. However, the material, thickness, number used during crimping, outer shape, structure, and the like of the sheet-like members are not particularly limited. The sheet-like members 451 and 452 may be provided with a structure having functions similar to those of the void portions 24a and the framework portions 24b included in the porous structure. That is, the sheet-like members may be formed of a porous structure. In a case where the sheet-like members are formed of a porous structure, the supply unit 420 functions as a supply unit for supplying the porous structure.

Operations of respective parts of the crimping device 400 can be controlled by a control unit including a CPU or the like. The crimping device 400 can also be configured to enable remote operation or the like in accordance with instructions from an external input device. For configurations of the crimping device 400 that are not particularly described, configurations of a known crimping device in a field of a stent manufacturing method can be arbitrarily adopted.

### <Crimping Method>

Next, a crimping method according to an embodiment (hereinafter also simply referred to as a “crimping method”) will be described with reference to FIGS. 11 to 15.

FIG. 11 is a flowchart illustrating respective steps of the crimping method.

The crimping method is a method for crimping the tubular stent 10, which is expandable in diameter, onto the balloon 220 using one or more pressing members 410 that are configured to be movable in the radially inward direction.

Specifically, as illustrated in FIG. 11, the crimping method includes arranging the stent 10 on the outer circumferential side of the balloon 220 (hereinafter referred to as an “arranging step S1”), reducing the diameter of the stent 10 by pressing the heated pressing members 410 against the porous structure 20 that covers the outer circumference of the stent 10, from the outer surface side thereof (hereinafter referred to as a “pressing step S2”), and pressurizing an inner cavity 225 of the balloon 220 at least once (hereinafter referred to as a “pressurizing step S3”).

Here, as the stent to be crimped, the stent 10 including the drug coating layer 18 described above (see FIG. 7) is exemplified. In addition, as the porous structure arranged between the stent 10 and the pressing members 410, the porous structure 20 forming the implantable device 100 described above is exemplified. In addition, as the crimping device that implements the crimping method, the crimping device 400 described above is exemplified.

Hereinafter, the crimping method will be described in detail.

Prior to setting the preparation item 100A in the crimping device 400, the arranging step S1 is performed. In the arranging step S1, the stent 10 is arranged on the outer circumferential side of the balloon 220. Specifically, as illustrated in FIG. 13, the balloon 220 is arranged on the inner circumferential side (a central portion side in the radial direction) of the plurality of pressing members 410, and the stent 10 is arranged to cover the outer circumference of the balloon 220.

In the preparation item 100A, at least a part of the porous structure 20 is fixed in advance to the stent 10 by the fixing portion 30 described above (see FIG. 6). Accordingly, the stent 10 is set in the crimping device 400 in a state in which the porous structure 20 is fixed to the stent 10.

The timing at which the porous structure 20 is set in the crimping device 400 is not particularly limited. For example, the porous structure 20 may be arranged on the outer circumferential side of the stent 10 after the balloon 220 and the stent 10 are set in the crimping device 400.

In a state in which the preparation item 100A is set in the crimping device 400, the sheet-like members 451 and 452 are arranged between the porous structure 20 and the pressing members 410. Here, two sheet-like members 451 and 452 are used, and the preparation item 100A is pinched between the respective sheet-like members 451 and 452. The crimping device 400 applies a predetermined tension to the respective sheet-like members 451 and 452 in a state in which the preparation item 100A is pinched between the respective sheet-like members 451 and 452.

Next, the pressing step S2 is performed. As illustrated in FIG. 13, a plurality of pressing members 410 heated to a predetermined temperature are moved in the radially inward direction to press the porous structure 20 from the outer surface side. Since the sheet-like members 451 and 452 are used, the pressing members 410 are pressed against the preparation item 100A from the outer surface side of the porous structure 20 with the sheet-like members 451 and 452 interposed therebetween.

In the pressing step S2, the timing at which heating of the pressing members 410 is started is not particularly limited. For example, heating may be started immediately before the pressing members 410 are moved toward the porous structure 20, or the pressing members 410 may be heated in advance before the stent 10 and the porous structure 20 are set in the crimping device 400.

A heating temperature of the pressing members 410 in the pressing step S2 can be set, for example, to be higher than 25° C. and lower than 55° C.

In a case where the balloon 220 is formed of an organic polymer material, a lower limit value of the heating temperature of the pressing members 410 is preferably equal to or higher than a glass transition temperature of a material forming the balloon 220. As an example, in a case where the balloon 220 is formed of a polymer material such as a polyamide elastomer, polyurethane, or a polyurethane elastomer, the lower limit value of the heating temperature can be set to a temperature higher than a glass transition temperature of each polymer material and a general room temperature (25° C.). That is, the lower limit value of the heating temperature can be set to a temperature higher than 25° C. This makes it possible to soften the balloon 220 more than in a case where heating is not performed when the pressing members 410 are heated, and thus the profile of the balloon 220 is reduced.

In a case where the porous structure 20 is formed of a polymer as described above, the lower limit value of the heating temperature of the pressing members 410 is preferably equal to or higher than a glass transition temperature of a material forming the porous structure 20. As an example, in a case where the porous structure 20 is formed of an aliphatic polyester (preferably polyglycolic acid (PGA)), the lower limit value of the heating temperature can be set to 40° C., which is the glass transition temperature. This allows an embedded state between the porous structure 20 and the stent 10 to be increased when the pressing members 410 are heated compared to a case where heating is not performed, and thus an outer diameter profile of the preparation item 100A is reduced.

In a case where the stent 10 is provided with the drug coating layer 18, an upper limit value of the heating temperature of the pressing members 410 is preferably a temperature that does not significantly affect elution of the drug coating layer 18. In a case where the stent 10 is provided with the drug coating layer 18 formed of the above-described material, the upper limit value of the heating temperature can be set to, for example, 54° C.

During the pressing step S2, the pressurizing step S3 can be performed at least once. By performing the pressurizing step S3, the balloon 220 expands in a radially outward direction, and a part of the balloon 220 enters gaps of the stent 10 (gaps formed between the strut portions 14, 15, and 16, and the curved portions 17). Accordingly, it is possible to improve a crimping force for holding the stent 10 with respect to the balloon 220 while reducing the profile of the implantable device 100 after crimping. The pressurizing step S3 may be performed when the pressing step S2 is not performed. The timing of starting the pressurizing step S3, the number of times the pressurizing step S3 is performed, and the pressure of the inner cavity 225 of the balloon 220 during pressurization can be arbitrarily set in accordance with product specifications of the implantable device 100 or the like.

The stent 10 has the drug coating layer 18 arranged on the outer surface 10b among the surfaces of the stent 10, the outer surface 10b facing the porous structure 20. Therefore, in the pressing step S2, when the pressing members 410 are pressed from the outer surface side of the porous structure 20, the recesses 18a and the protrusions 18b corresponding to the porous structure 20 are formed in the drug coating layer 18 (see FIG. 8). Specifically, in the drug coating layer 18, the recesses 18a having a recessed shape deeper than surrounding portions are formed at portions corresponding to the framework portions 24b of the porous structure 20 (portions where the framework portions 24b are pressed). In addition, in the drug coating layer 18, protrusions 18b that protrude more than the recesses 18a are formed at portions corresponding to the void portions 24a of the porous structure 20. Accordingly, since the porous structure 20 is in a state of being embedded in the drug coating layer 18, resistance to positional displacement of the porous structure 20 in the circumferential direction or the axial direction from a state in which the porous structure 20 is arranged on the stent 10 becomes greater than in a state in which the porous structure 20 is not embedded in the drug coating layer 18.

In addition, in the above-described crimping method, the pressing step S2 is performed in a state in which the porous structure 20 is arranged between the pressing members 410 and the stent 10. By arranging the porous structure 20 between the pressing members 410 and the stent 10, it is possible to increase frictional force produced between the pressing members 410 and the porous structure 20 when the stent 10 is crimped onto the balloon 220, as compared with a case in which the pressing members 410 are directly pressed against the stent 10. This effect of increasing the frictional force is achieved because a coarseness of the framework portions 24b of the porous structure 20 is smaller than a coarseness of the stent 10. By arranging the porous structure 20 between the pressing members 410 and the stent 10 as described above and performing the pressing step S2 in a state in which a greater frictional force is produced between the pressing members 410 and the porous structure 20, it is possible to prevent the porous structure 20 from slipping on the stent 10 and being displaced when the pressing force is applied from the pressing members 410 to the sheet-like members 451 and 452. Accordingly, the pressing members 410 can apply the pressing force uniformly to respective portions of the stent 10 in the circumferential direction.

In addition, when the pressing force is applied from the pressing members 410 to the porous structure 20 in the pressing step S2, the porous structure 20 reduces its diameter uniformly along the circumferential direction by reducing gaps of the void portions 24a of the porous structure 20 or by moving the framework portions 24b so that overlap between the void portions 24a increases. At this time, since the porous structure 20 is in a state of being embedded in the drug coating layer 18 and starts reducing its diameter in the circumferential direction from a state in which frictional force between the porous structure 20 and the stent 10 is increased, the diameter is uniformly reduced along the circumferential direction while maintaining intervals between struts (specifically, between the first strut portions 15 and the second strut portions 16, and between the first strut portions 15 and the third strut portions 14). Accordingly, when a pressing force is applied from the pressing members 410 to the porous structure 20, the porous structure 20 reduces its diameter uniformly along the circumferential direction while applying a uniform pressing force to respective portions of the stent 10 in the circumferential direction arranged on the inner circumferential side of the porous structure 20. As a result, it becomes less likely that any interval between the struts becomes excessively narrow, and it is possible to prevent a force that pinches the balloon 220 from becoming excessively strong. Accordingly, in the above-described crimping method, formation of pinholes in the balloon 220 can be effectively prevented in the pressing step S2.

The pressing step S2 is performed in a state in which the pressing members 410 are heated. Accordingly, the porous structure 20 is pressed against the drug coating layer 18 arranged on the outer surface 10b of the stent 10 by the heated pressing members 410. The drug coating layer 18 is softened by heat transmitted from the pressing members 410. As a result, at least a part of the porous structure 20 is arranged to be more deeply embedded in the drug coating layer 18 than in a state in which heating is not performed by the pressing step S2. This increases resistance to positional displacement between the porous structure 20 and the stent 10 in the circumferential direction or the axial direction compared to a state in which heating is not performed, and enhances an effect of uniformly reducing intervals between the struts (specifically, between the first strut portions 15 and the second strut portions 16, and between the first strut portions 15 and the third strut portions 14) along the circumferential direction. Accordingly, it becomes even less likely that an interval at any location between the struts becomes excessively narrow during the pressing step S2, and it is possible to prevent a force that pinches the balloon 220 from becoming excessively strong. Therefore, formation of pinholes in the balloon 220 can be more effectively prevented.

In addition, since the pressing step S2 is performed in a state in which the pressing members 410 are heated as described above, the balloon 220 is also softened together with the drug coating layer 18 by heat transmitted from the pressing members 410. Accordingly, in the balloon 220, gaps between folded portions 227 of the balloon 220 become smaller, and intervals between the folded portions 227 become denser due to a diameter-reducing force applied from the pressing members 410 during the pressing step S2. Therefore, the profile of the balloon 220 is effectively reduced after the pressing step S2.

In addition, the pressing step S2 with respect to the preparation item 100A is performed in a state in which the sheet-like members 451 and 452 are arranged between the porous structure 20 and the pressing members 410. The sheet-like members are made of a polymer film. The sheet-like members 451 and 452 function as buffer members for forming a space defined inside the pressing ends 411 into a shape closer to a circular shape when the pressing ends 411 of the respective pressing members 410 come into contact with the preparation item 100A as the respective pressing members 410 move in the radially inward direction. For example, as illustrated in FIGS. 13 to 15, when the pressing ends 411 come into contact with the preparation item 100A in a state in which a space defined inside the pressing ends 411 of the respective pressing members 410 (a space in which the preparation item 100A is set) is formed into a polygonal shape in accordance with a cross-sectional shape of the pressing ends 411, gaps that do not contact the preparation item 100A are formed at portions positioned at corners (vertices) of the polygon. If the pressing step S2 proceeds in a state in which such gaps are formed, it becomes impossible to appropriately apply a pressing force to an entire range of the preparation item 100A in the circumferential direction, and it becomes impossible to apply a pressing force to the preparation item 100A along a circular cross-section. As a result, the profile of the implantable device 100 cannot be sufficiently reduced. By performing the pressing step S2 in a state in which the sheet-like members 451 and 452 are arranged between the pressing members 410 and the preparation item 100A, it is possible to reduce an influence of gaps formed between the preparation item 100A and the pressing ends 411 of the pressing members 410 as described above. Accordingly, the profile of the implantable device 100 after the pressing step S2 can be effectively reduced.

After the profile of the implantable device 100 reaches a predetermined size by performing the pressing step S2 and the pressurizing step S3 (at least once), the pressing members 410 are moved in the radially outward direction. By moving the pressing members 410 in the radially outward direction, application of the pressing force to the implantable device 100 is released. The crimping method can be completed by the above procedure. In addition, by manufacturing the implantable device 100 by the crimping method described above, a manufacturing method for the balloon catheter 200 (the stent delivery system 300) including components other than the implantable device 100 is provided.

As described above, the crimping method is a crimping method of crimping the tubular stent 10 that is expandable in diameter onto the balloon 220 using one or more pressing members 410 that are configured to be movable in the radially inward direction, the method including arranging the stent 10 on the outer circumference of the balloon 220, reducing the diameter of the stent 10 by pressing the heated pressing members 410 against the porous structure 20 that covers the outer circumference of the stent 10, from the outer surface side thereof, and pressurizing the inside of the balloon 220 at least once.

By reducing the diameter of the stent 10 by pressing the heated pressing members 410 against the porous structure 20 from the outer surface side thereof in a state in which the porous structure 20 is arranged to cover the outer circumference of the stent 10, and further pressurizing the inside of the balloon 220 at least once, it is possible to improve a crimping force of the implantable device 100 and reduce the profile of the implantable device 100 while reducing the occurrence rate of pinholes.

In addition, the implantable device 100 is an implantable device arranged on the outer circumference of the balloon 220 that is expandable and contractible, the implantable device including the tubular stent 10 that is expandable in diameter and is crimped onto the outer circumferential surface of the balloon 220, and the porous structure 20 that is arranged to cover the stent 10 and of which at least a part is fixed to the stent 10, in which the stent 10 has the drug coating layer 18 arranged on the outer surface 10b among surfaces of the stent 10, the outer surface 10b facing the porous structure 20, the porous structure 20 includes the void portions 24a penetrating through the porous structure 20 in the thickness direction and the framework portions 24b that define the void portions 24a, and the drug coating layer 18 has the recesses 18a and the protrusions 18b corresponding to the void portions 24a and the framework portions 24b of the porous structure 20.

According to the implantable device 100 configured as described above, when the stent 10 is crimped onto the balloon 220, the porous structure 20 is pressed against the drug coating layer 18 so as to form the recesses 18a, thereby allowing a uniform pressing force to be applied to the stent 10 in the circumferential direction. Accordingly, the implantable device 100 can improve a crimping force and reduce a profile while reducing the occurrence rate of pinholes.

The crimping device 400 includes one or more pressing members 410 that crimp the stent 10 onto the balloon 220 arranged on the inner circumferential side of the stent 10 by reducing the diameter of the stent 10 in accordance with movement in the radially inward direction, and the supply unit 420 that supplies one or more porous structures (for example, sheet-like porous structures) between the pressing members 410 and the stent 10.

According to the crimping device 400 configured as described above, when a pressing force is applied from the pressing members 410 to a sheet-like porous structure in the pressing step S2, the porous structure on the sheet reduces its diameter uniformly along the circumferential direction by reducing gaps of void portions of the porous structure on the sheet or by moving framework portions so that overlap between the void portions increases. Accordingly, when the pressing force is applied from the pressing members 410 to the sheet-like porous structure, the sheet-like porous structure reduces its diameter uniformly along the circumferential direction while applying a uniform pressing force to respective portions of the stent 10 in the circumferential direction arranged on the inner circumferential side of the sheet-like porous structure. As a result, it becomes less likely that any interval between the struts becomes excessively narrow, and it is possible to prevent a force that pinches the balloon 220 from becoming excessively strong. Accordingly, in the crimping method using the crimping device 400, by supplying the sheet-like porous structure from the supply unit 420, it is possible to effectively prevent formation of pinholes in the balloon 220 in the pressing step S2.

### MODIFICATION EXAMPLE

The above-described crimping method can be modified as long as it includes “reducing a diameter of a stent by pressing heated pressing members against a porous structure from an outer surface side of the porous structure in a state in which the porous structure is arranged so as to cover an outer circumference of the stent, and pressurizing an inside of the balloon at least once”. For example, the following modification examples can be adopted.

In the above-described embodiments, an example has been described in which, when pressing is performed by the pressing members 410, the porous structure 20 used as a component of the implantable device 100 is arranged so as to cover the outer circumference of the stent 10. However, the porous structure used in the crimping method is not limited to the porous structure 20 as described above. For example, the porous structure may be formed of a sheet-like member supplied from the crimping device 400 when pressing by the pressing members 410 is started. That is, a sheet-like member provided with a structure having functions similar to those of the void portions 24a and the framework portions 24b may be formed as the porous structure, and pressing by the pressing members 410 can be performed in a state in which the stent 10 is covered with this porous structure. Accordingly, the crimping method according to the modification example can include a configuration “the porous structure is formed of sheet-like members, and pressing members press the porous structure from an outer surface side in a state in which a stent is pinched between a plurality of the sheet-like members”.

Even in a case where the porous structure formed of the sheet-like members as described above is used, it is possible to achieve effects such as “preventing formation of pinholes in the balloon 220 by performing the pressing step S2 in a state in which a greater frictional force is produced between the pressing members 410 and the porous structure” and “in the pressing step S2, reducing a diameter of the porous structure uniformly along the circumferential direction by reducing gaps of void portions of the porous structure or by moving framework portions, thereby applying a uniform pressing force to respective portions of the stent 10 in the circumferential direction arranged on an inner circumferential side of the porous structure, and thus effectively preventing formation of pinholes in the balloon 220” as described above.

The porous structure may also be formed of, for example, a tubular member having an inner cavity. As one example, a member similar to the tubular porous structure used as a component of the implantable device 100 can be used as a jig when pressing is performed by the pressing members 410. Accordingly, the crimping method according to this modification example can include a configuration “the porous structure is formed of a tubular member having an inner cavity, and pressing members press the porous structure from an outer surface side in a state in which a stent is inserted into the inner cavity of the porous structure”.

Even in a case where the porous structure configured as described above is used, it is possible to achieve effects similar to those of the crimping method using the porous structure formed of sheet-like members.

In a case where the porous structure described in the above modification example is used, the porous structure is not used as a component of the implantable device. Accordingly, the implantable device to which the crimping method using the porous structures described in the modification example is applied is formed of the balloon 220 and the stent 10 crimped onto the balloon 220.

As described above, the crimping method, the manufacturing method for the balloon catheter, the implantable device, and the crimping device have been described through the embodiments and the modification examples, but the present disclosure is not limited to the contents described in the specification and can be appropriately modified.

For example, although a stent provided with a drug coating layer has been exemplified in the above description, the stent to which the crimping method, the manufacturing method for the balloon catheter, and the crimping device according to the present disclosure are applied is not limited to one provided with the drug coating layer.

What is claimed is:

1. A method for crimping a tubular stent onto a balloon using one or more pressing members, the method comprising:
arranging the stent on an outer circumferential side of the balloon;
heating the one or more pressing members;
reducing a diameter of the stent by pressing the one or more heated pressing members against a porous structure that covers an outer circumference of the stent, from an outer surface side of the porous structure; and
pressurizing an inside of the balloon at least once.

2. The method according to claim 1, wherein
a drug coating layer is arranged on a surface of the stent that faces the porous structure, and
the one or more heated pressing members are pressed against the porous structure to form, on the drug coating layer, recesses and protrusions corresponding to the porous structure.

3. The method according to claim 1, wherein
the one or more pressing members are heated to a temperature higher than 25° C. and lower than 55° C.

4. The method according to claim 1, further comprising:
fixing at least a part of the porous structure to the stent before arranging the stent on the outer circumferential side of the balloon.

5. The method according to claim 4, wherein
sheet-like members are arranged between the porous structure and the one or more pressing members, and
the one or more heated pressing members are pressed from an outer surface side of the sheet-like members and the outer surface side of the porous structure.

6. The method according to claim 1, wherein
the porous structure is arranged to cover the outer circumference of the stent while the stent is arranged on the outer circumferential side of the balloon.

7. The method according to claim 6, wherein
the porous structure is formed of sheet-like members, and
the one or more heated pressing members are pressed against the porous structure from the outer surface side of the porous structure while the stent is pinched between a plurality of sheet-like porous structures.

8. The method according to claim 6, wherein
the porous structure is formed of a tubular member including an inner cavity, and
the one or more heated pressing members are pressed against the porous structure from the outer surface side of the porous structure while the stent is inserted into the inner cavity of the porous structure.

9. A method for manufacturing a balloon catheter, the method comprising:
crimping a tubular stent onto a balloon of the balloon catheter by the method according to claim 1.

10. An implantable device comprising:
a radially expandable tubular stent that is crimped onto an outer circumferential surface of a balloon; and
a porous structure arranged to cover the stent, wherein
at least a part of the porous structure is fixed to the stent,
the porous structure includes void portions penetrating through the porous structure in a thickness direction and framework portions that define the void portions,
the stent includes a drug coating layer on a surface of the stent that faces the porous structure, and
the drug coating layer has recesses and protrusions corresponding to the void portions and the framework portions.

11. The implantable device according to claim 10, wherein
at least a part of the porous structure is embedded in the recesses of the drug coating layer.

12. The implantable device according to claim 10, wherein
the porous structure is formed of a metal or a polymer.

13. The implantable device according to claim 10, wherein
the stent includes linear rings that form an outer circumference of a cylindrical shape in which gaps are formed, and a plurality of link portions that connect the linear rings to each other in the gaps,
the drug coating layer is not formed on the link portions, and
the porous structure is fixed to at least the link portions.

14. The implantable device according to claim 10, wherein
the porous structure has a cylindrical shape extending in an axial direction of the stent.

15. The implantable device according to claim 10, wherein
the stent includes linear rings that form an outer circumference of a cylindrical shape, and
a size of each of the void portions is smaller than a gap between adjacent ones of the linear rings.

16. The implantable device according to claim 10, wherein
the porous structure is formed of a knitted fabric, a woven fabric, or a molded product in which slits or holes are formed.

17. The implantable device according to claim 10, further comprising:
a fixing portion that fixes the porous structure to the stent, wherein the fixing portion is formed of a biodegradable polymer.

18. The implantable device according to claim 10, wherein
the drug coating layer includes a drug supported by a biodegradable polymer.

19. A device for crimping a tubular stent that is radially expandable onto a balloon, the device comprising:
one or more pressing members that crimp the stent onto the balloon by reducing a diameter of the stent in accordance with movement of the one or more pressing members in a radially inward direction, the balloon being arrangeable on an inner circumferential side of the stent; and
one or more supply rollers configured to supply one or more porous structures between the one or more pressing members and the stent.

20. The device according to claim 19, further comprising:
a heater configured to heat the one or more pressing members.
